(19) United States
(12) Patent Application Publication
(10) Pub. No.: US 2021/0213223 A1
(43) Pub. Date: Jul. 15, 2021

(54) Medical mechanical controlled balance ventilation single-lumen lung isolation catheter bridging spans trachea to bronchus and by-pass flow gas holes of wall

(71) Applicant: Jinfang XIAO, Guangdong (CN)

(72) Inventors: Jinfang XIAO, Guangdong (CN); Ming XIAO, Guangdong (CN)

(21) Appl. No.: 16/092,448

(22) PCT Filed: May 5, 2017
(86) PCT No.: PCT/CN2017/083268

§ 371 Date: Oct. 10, 2018

(30) Foreign Application Priority Data
Apr. 18, 2017 (CN) 201710252634.X

(51) Int. Cl. A61M 16/04; A61M 25/00

(52) U.S. Cl. CPC A61M 16/0434; A61M 2210/1035; A61M 25/007

(57) ABSTRACT

A medical mechanical controlled balance ventilation single-lumen lung isolation catheter bridging spans trachea to bronchus and by-pass flow gas holes of wall, which has a intraluminal main trachea tube and a distal intraluminal bronchus tube in communication with one end of the intraluminal main trachea tube; a first opening is provided at a rear end of the distal intraluminal bronchus tube; a second holes opening zone having at least two holes is provided at a side wall of a lower end of the intraluminal main trachea tube; the transmural holes of the second holes opening zone are distributed along a same or different horizontal sectional surface or vertical sectional surface of a three dimensional space formed inside the intraluminal main trachea tube.

### CROSS REFERENCING OF RELATED APPLICATIONS

The present invention claims the priority of Chinese patent application number 201710252634.X (CN106938115A) filed on Apr. 18, 2017 having the same title of invention as the present application, and the entire contents thereof are incorporated herein by reference.

### BACKGROUND OF THE INVENTION

The present invention relates to the field of medical apparatus, and more specifically relates to a medical mechanical controlled balance ventilation single-lumen lung isolation catheter bridging spans trachea to bronchus and by-pass flow gas holes of wall.

Double-lumen endotracheal tube has been used clinically for 40 years without any technical breakthrough. The tube is wide and thick due to an “isolation wall” in the middle of the tube. The main function of the “isolation wall” is to separate the left lung and the right lung. Also, the “isolation wall” ensures balanced ventilation between the left lung and the right lung. In short, the “isolation wall” causes the tube to be wide and thick. Such a tube can damage the oral cavity, the vocal cord, and the tracheal wall of a patient. Use of such a tube requires alignment which may easily fail and thus requires a more sophisticated apparatus like a fiberoptic bronchoscope to help with the alignment. When a patient changes his/her body position, the tube may easily rotate and displace and thus cannot meet the requirements for minimally invasive surgery for visualized cardiothoracic surgery in children. Most importantly, the tube may easily cause complete lung collapse or lung deflation at the surgical site, increase the possibility of acute lung injury, recurrent pulmonary edema, and general lung infections after surgery. Further, the tube has multiple connectors which complicate the preparation procedures for anaesthesia and increase the risk of loosen connectors.

In summary, the double-lumen endotracheal tube of the prior art has the following ten major disadvantages: 1. The tube is wide and thick and susceptible of damaging oral tissues, vocal cord and tracheal wall of a patient, and the mouth and pharyngeal cavity of the patient has to be sufficiently exposed; 2. The tube may easily fail to be aligned and therefore requires a fiberoptic bronchoscope to help with the alignment; 3. When a body position of the patient changes, the tube may easily rotate and displace, thereby affecting ventilation and risking the patient's life; 4. Manufacturing of the tube is complicated and thus involves a high cost; 5. High consumption of raw materials, which is not environmentally friendly; 6. High price, which in turns increase the overall medical expenses; 7. Most importantly, the tube may easily cause complete lung collapse or lung deflation at the surgical site, increase the possibility of acute lung injury, recurrent pulmonary edema, and general lung infections after surgery; 8. The tube is complicated to use, and the requirements for insertion under non-ventilated condition increases the risk of Iatrogenic hypoxia of the patient; 9. The tube does not possess the characteristics of unbalanced air ventilation to the left lung and the right lung during anatomical alignment; 10. A bridge structure cannot be spanned in the trachea and therefore causes air advection flow in the lung which is one of the factors for normal ventilator induced lung injury.

### BRIEF SUMMARY OF THE INVENTION

The present invention provides a medical mechanical controlled balance ventilation single-lumen lung isolation catheter bridging spans trachea to bronchus and by-pass flow gas holes of wall, to substitute the conventional double-lumen endotracheal tube. The present invention solves the problems of overly wide diameter of the double-lumen endotracheal tube of the prior art, prevents failure of use because of easy damaging due to bending in a prior art lung isolation tube, and also prevents the effect of use influenced by relatively scattered ventilation air flow at the zone of a second hole opening area.

A medical mechanical controlled balance ventilation single-lumen lung isolation catheter bridging spans trachea to bronchus and by-pass flow gas holes of wall, comprising a intraluminal main trachea tube and a distal intraluminal bronchus tube in communication with one end of the intraluminal main trachea tube; a first opening is provided at a rear end of the distal intraluminal bronchus tube; a second holes opening zone having at least two holes is provided on wall of a lower end of the intraluminal main trachea tube; the holes of the second holes opening zone are distributed along the same or different horizontal sectional surface or vertical sectional surface of a three dimensional space formed inside the intraluminal main trachea tube.

Optionally, when the second holes opening zone has three holes, the three holes are arranged to define a triangular shape within the three dimensional space formed by an inner side wall of the intraluminal main trachea tube.

Specifically, when there are three holes in the second holes opening zone, the three holes are arranged to define a triangular shape within the three dimensional space formed on wall of the intraluminal main trachea tube; the triangular shape as defined is any one of an isosceles triangle, equilateral triangle, right angled triangle, right angled isosceles triangle or right angled triangle having one included angle of distribution being 30 degrees on wall of tube, or any triangle that has 180 degrees as a sum of all included angles of distribution on wall of tube.

Optionally, when there are four holes on wall of the intraluminal main trachea tube in the second holes opening zone, the four holes are arranged to define a parallelogram shape or any quadrilateral having 360 degrees being a sum of all included angles within the three dimensional space formed on wall of the intraluminal main trachea tube. The shape as defined has a pentagonal shape when there are five holes, and the shape as defined has a hexagonal shape when there are six holes, and so and so forth.

Optionally, when there are four holes on wall of the intraluminal main trachea tube in the second holes opening zone, the four holes divided into two pairs with each pair of holes opposing each other define a cross shape within the three dimensional space formed by an inner side wall of the intraluminal main trachea tube.

Specifically, when there are four holes on wall of the intraluminal main trachea tube in the second holes opening zone, the four holes divided into two pairs with each pair of holes opposing each other define a cross shape within the three dimensional space formed on wall of the intraluminal main trachea tube, and the cross shape has equal width and height.

In actual use, each of the holes on wall of the intraluminal main trachea tube of the second holes opening zone is an inclined hole through the wall of the intraluminal main trachea tube; a wall of each hole is inclined with respect to a perpendicular position to a side wall of the intraluminal main trachea tube.

Specifically, each hole is inclined with respect to a perpendicular position through the wall of the intraluminal main trachea tube, and an angle through the wall of the intraluminal main trachea tube inclination is 0-90 degrees.

Specifically, the angle through the wall of the intraluminal main trachea tube inclination comprises any one of 5, 10, 15, 30, 45 and 60 degrees.

In actual use, a suction hole is provided at a lower part of the intraluminal main trachea tube proximal to the distal intraluminal bronchus tube; the suction hole faces away from the first opening; a hole diameter of the suction hole is the same as a diameter of the first opening.

Specifically, a suction catheter is inserted into the suction hole; two small holes are provided on the suction catheter; the two small holes are opposed to each other.

Specifically, one end of the suction catheter is bent, and an angle of bending is 90-150 degrees.

Further, the suction catheter has a diameter of one third of a diameter of the distal intraluminal bronchus tube.

In actual use, an outer wall of the intraluminal main trachea tube at a position above the second holes opening zone is provided with the tracheal cuff; the tracheal cuff is in communication with a first inflating valve; an outer wall of the distal intraluminal bronchus tube is provided with the bronchial cuff; the bronchial cuff is in communication with a second inflating valve; the distal intraluminal balloon blocker is provided inside the distal intraluminal bronchus tube; the distal intraluminal balloon blocker is in communication with a third inflating valve via a ventilation pressure controller; the ventilation pressure controller indicates pressure level inside the distal intraluminal balloon blocker and adjusts a size of the distal intraluminal balloon blocker for blocking, such that an amount of ventilated air flow at the distal intraluminal bronchus tube is regulated and the balanced ventilation between the left and right lungs is regulated.

The first opening has an area S_{end}; a total area of the holes in the second holes opening zone is S; wherein S≥1.5S_{end}.

Compared with the prior arts, the present invention has the following advantages:

The present invention provides a medical mechanical controlled balance ventilation single-lumen lung isolation catheter bridging spans trachea to bronchus and by-pass flow gas holes of wall, comprising a intraluminal main trachea tube and a distal intraluminal bronchus tube in communication with one end of the intraluminal main trachea tube; a first opening is provided at a rear end of the distal intraluminal bronchus tube; a second holes opening zone having at least two holes is provided on wall of a lower end of the intraluminal main trachea tube; the holes of the second holes opening zone are distributed along the same or different horizontal sectional surface or vertical sectional surface of a three dimensional space formed inside the intraluminal main trachea tube. Because the holes of the second holes opening zone provided on wall of the lower end of the intraluminal main trachea tube are distributed along the same or different horizontal sectional surface or vertical sectional surface, the medical single-lumen lung isolation catheter can be prevented from damaging due to bending even without thickening the wall of the intraluminal main trachea tube.

In summary, the present invention has the following inventive features: 1) more than 2 holes are provided at a lower end portion of the intraluminal main trachea tube; orientation and arrangement of the holes are designed in accordance with physics and thus beneficial to balanced ventilation and mechanical strength of the associated part; 2) an electronic ventilation pressure controller can indicate the pressure of the distal intraluminal balloon blocking piece for blocking inside the trachea and at the outer wall of the trachea, and can regulate the size of the interior air cell blocking piece; 3) the distal intraluminal balloon blocking piece which can be regulated is provided in the single lumen of the intraluminal main trachea tube and the distal intraluminal bronchus tube to regulate the amount of ventilated air in the distal intraluminal bronchus tube and the balanced ventilation between the left lung and the right lung; 4) By adjusting the size of the balloon of tracheal cuff and the balloon of bronchial cuff, bridge structure spans are defined by the bulged portions of the air cells between the trachea and the bronchus and the tube of the present invention; multiple holes changes air advection according to aerodynamics and thus resulting in the gas flow resembling more to physiological mixed flow of air during physiological ventilation; 5) The present invention first finds the mechanism of catheter with controlled balanced ventilation and bridge span from trachea to bronchus in patients to ensure minimal effective ventilation in medical single-lumen ventilation so as to reduce tracheal injury, simplify alignment, and solve the problem of lung isolation in children; controlled balanced ventilation can effectively regulate oxygen supply and consumption, specifically the oxygen supply of COPD (chronic obstructive pulmonary disease) surgery in the elderly, and solves the problem of easy lack of oxygen in children, and improves pulmonary artery hypertension prior and subsequent to lung deflation.

### BRIEF DESCRIPTION OF THE DRAWINGS

In order to illustrate more clearly the technical solution of the prior arts and of the embodiments of the medical mechanical controlled balance ventilation single-lumen lung isolation catheter bridging spans trachea to bronchus and by-pass flow gas holes of wall according to the present invention, the drawings necessary to illustrate the embodiments and the prior arts are briefly described below. Obviously, the drawings as described only illustrate some specific embodiments of the present invention. Other drawings obtainable without any inventive effort by a person skilled in this field of art based on the teachings of the drawings herein should also fall within the scope of protection of the present invention.

FIG. 1 is a structural view of a medical mechanical controlled balance ventilation single-lumen lung isolation catheter bridging spans trachea to bronchus and by-pass flow gas holes of wall according to the present invention.

FIGS. 2(a), 2(b) and 2(c) are perspective and vertical sectional views showing a first embodiment of hole distribution of the second holes opening zone according to an embodiment of the present invention.

FIGS. 3(a), 3(b) and 3(c) are perspective and vertical sectional views showing a second embodiment of hole distribution of the second holes opening zone according to an embodiment of the present invention.

FIGS. 4(a), 4(b) and 4(c) are perspective and vertical sectional views showing a third embodiment of hole distribution of the second holes opening zone according to an embodiment of the present invention.

FIGS. 5(a), 5(b) and 5(c) are perspective and vertical sectional views showing a fourth embodiment of hole distribution of the second holes opening zone according to an embodiment of the present invention.

FIGS. 6(a), 6(b) and 6(c) are perspective and vertical sectional views showing a fifth embodiment of hole distribution of the second holes opening zone according to an embodiment of the present invention.

FIGS. 7(a), 7(b) and 7(c) are perspective and vertical sectional views showing a sixth embodiment of hole distribution of the second holes opening zone according to an embodiment of the present invention.

FIGS. 8(a), 8(b) and 8(c) are perspective and vertical sectional views showing a seventh embodiment of hole distribution of the second holes opening zone according to an embodiment of the present invention.

FIGS. 9(a), 9(b) and 9(c) are perspective and vertical sectional views showing an eighth embodiment of hole distribution of the second holes opening zone according to an embodiment of the present invention.

FIG. 10(a)-FIG. 10(f) are sectional views showing different possible angles of inclination of a hole of the second holes opening zone according to the present invention.

FIG. 11 shows the structure of a suction catheter specifically to be used with the present invention.

FIG. 12 is a diagram showing the use of the present invention.

FIG. 13 is another diagram showing the use of the present invention.

FIG. 14 shows the bridge structure spans and balanced lung ventilation of the present invention during use.

### REFERENCES IN THE FIGURES

1: intraluminal main trachea tube; 2: distal intraluminal bronchus tube; 21: first opening; 11: second holes opening zone; 12: suction hole; 3: suction catheter; 31: small holes; 4: tracheal cuff; 41: first inflating valve; 5: bronchial cuff; 51: second inflating valve; 6: distal intraluminal balloon blocker; 61: third inflating valve; 62: ventilation pressure controller.

### DETAILED DESCRIPTION OF THE INVENTION

The present invention will be clearly and comprehensively described below with reference to the drawings. Obviously, the embodiments described as follows are only part but not all of the embodiments of the present invention. All other embodiments obtainable without any inventive effort by a person skilled in this field of art based on the teachings of the embodiments of the present invention should fall within the scope of protection of the present invention.

It should be noted that, in the following description, terms like “center”, “upper”, “lower”, “left”, “right”, “vertical”, “horizontal”, “inside” and “outside” that indicate directions or positional relationships should be understood according to the directions or positional relationships as shown in the drawings, and they are intended only for convenient and simplified description of the present invention, and should not be understood to bear any indication or implication of limiting the device or components as described to be necessarily being oriented, structured and operated according to the specifically described directions, and so they should not limit the present invention. Also, terms like “first”, “second” and third” are intended only for illustration and have no indication or implication regarding the relative importance of the elements being described.

It should be noted that, in the following description of the present invention, terms like “installed”, “connected”, “communicated” should be interpreted broadly, unless otherwise specified and limited. For example, connection can be interpreted as fixed connection, removable connection or connection as a one whole piece, and it can be achieved mechanically as well as electrically, directly or indirectly through a medium, or it may refer to internal connection of two components. A person skilled in this field of art may interpret the specific meaning of the terms in a specific occurrence in the following description according to the specific context of the description.

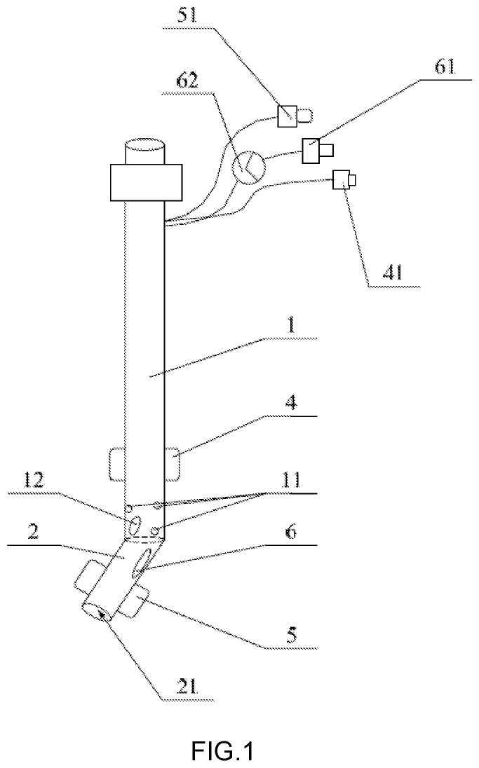

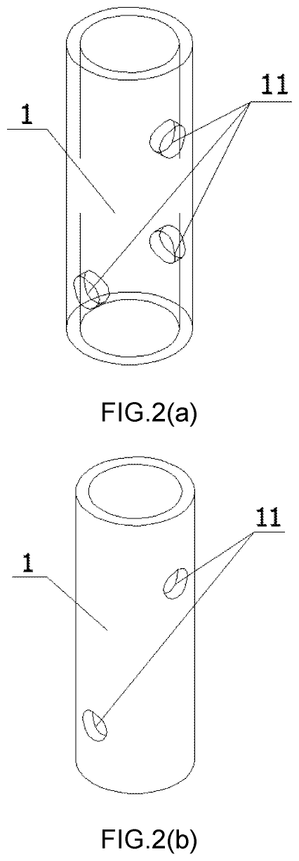

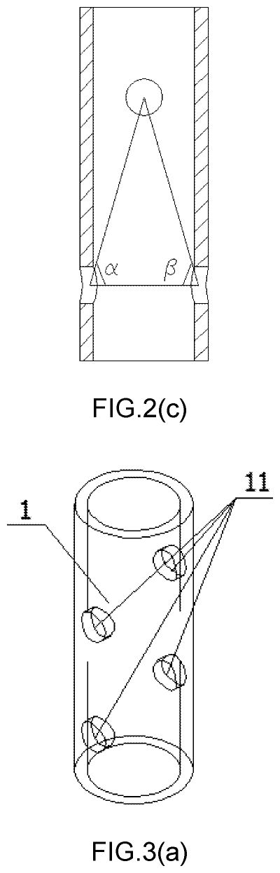

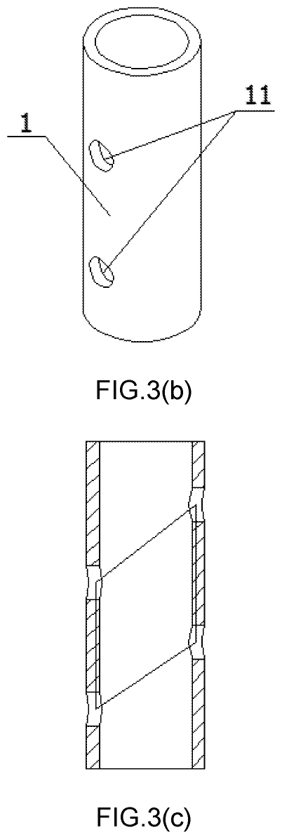

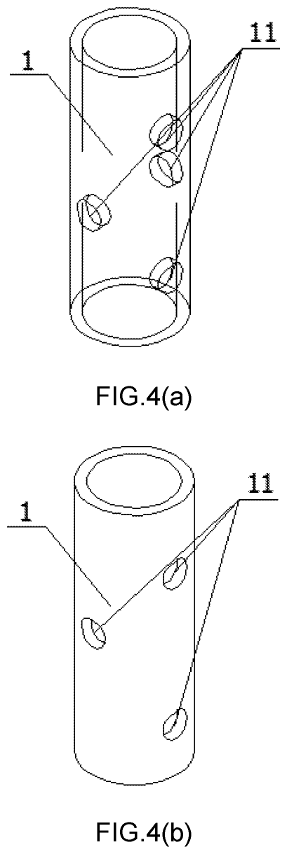

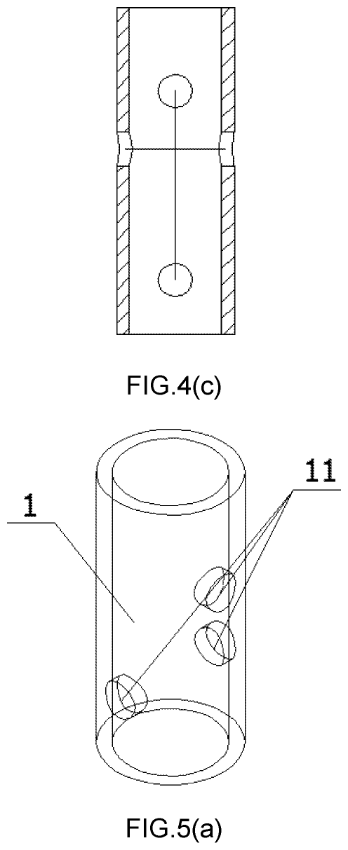

FIG. 1 is a structural view of the medical mechanical controlled balance ventilation single-lumen lung isolation catheter bridging spans trachea to bronchus and by-pass flow gas holes of wall according to the present invention. FIGS. 2(a), 2(b) and 2(c) are perspective and vertical sectional views showing a first embodiment of hole distribution of the second holes opening zone according to an embodiment of the present invention. FIGS. 3(a), 3(b) and 3(c) are perspective and vertical sectional views showing a second embodiment of hole distribution of the second holes opening zone according to an embodiment of the present invention. FIGS. 4(a), 4(b) and 4(c) are perspective and vertical sectional views showing a third embodiment of hole distribution of the second holes opening zone according to an embodiment of the present invention.

As shown in FIG. 1 and also FIG. 2(a)-FIG. 2(c), FIG. 3(a)-FIG. 3(c), FIG. 4(a)-FIG. 4(c), the present invention provides a medical mechanical controlled balance ventilation single-lumen lung isolation catheter bridging spans trachea to bronchus and by-pass flow gas holes of wall, comprising a intraluminal main trachea tube 1 and a distal intraluminal bronchus tube 2 in communication with one end of the intraluminal main trachea tube 1; a first opening 21 is provided at a rear end of the distal intraluminal bronchus tube 2; a second holes opening zone 11 having at least two holes is provided at a side wall of a lower end of the intraluminal main trachea tube 1; the holes of the second holes opening zone 11 are distributed along the same or different horizontal sectional surface or vertical sectional surface of a three dimensional space formed inside the intraluminal main trachea tube 1 (as shown in FIG. 2(a)-FIG. 2(c), FIG. 3(a)-FIG. 3(c), FIG. 4(a)-FIG. 4(c)).

Compared with the prior arts, the present invention has the following advantages:

As shown in FIG. 1 and also FIG. 2(a)-FIG. 2(c), FIG. 3(a)-FIG. 3(c), FIG. 4(a)-FIG. 4(c), the present invention provides a medical mechanical controlled balance ventilation single-lumen lung isolation catheter bridging spans trachea to bronchus and by-pass flow gas holes of wall, comprising a intraluminal main trachea tube 1 and a distal intraluminal bronchus tube 2 in communication with one end of the intraluminal main trachea tube 1; a first opening 21 is provided at a rear end of the distal intraluminal bronchus tube 2; a second holes opening zone 11 having at least two holes is provided at a side wall of a lower end of the intraluminal main trachea tube 1; the holes of the second holes opening zone 11 are distributed along the same or different horizontal sectional surface or vertical sectional surface of a three dimensional space formed inside the intraluminal main trachea tube 1 (as shown in FIG. 2(a)-FIG. 2(c), FIG. 3(a)-FIG. 3(c), FIG. 4(a)-FIG. 4(c)). Because the holes of the second holes opening zone 11 provided at the side wall of the lower end of the intraluminal main trachea tube 1 are distributed along the same or different horizontal sectional surface or vertical sectional surface, the single-lumen lung isolation catheter can be prevented from damaging due to bending even without thickening the wall of the intraluminal main trachea tube 1.

As shown in FIG. 2(a)-FIG. 2(c), when the second holes opening zone 11 has three holes, the three holes are arranged to define a triangular shape within the three dimensional space formed on wall of the intraluminal main trachea tube 1.

To ensure the amount of ventilated air while further improving the stability of the intraluminal main trachea tube 1, when there are three holes in the second holes opening zone 11 as shown in FIG. 2(a)-FIG. 2(c) in actual production, the three holes are arranged to define a triangular shape within the three dimensional space formed by an inner side wall of the intraluminal main trachea tube 1; the triangular shape as defined can be an isosceles triangle which means two of the three included angles thereof are the same; as illustrated in FIG. 2, angle α is the same as angle β.

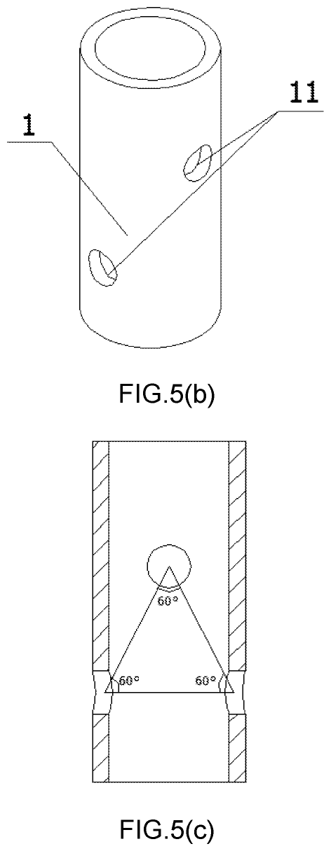

FIGS. 5(a), 5(b) and 5(c) are perspective and vertical sectional views showing a fourth embodiment of holes distribution of the second holes opening zone according to an embodiment of the present invention.

As shown in FIGS. 5(a), 5(b) and 5(c), when there are three holes in the second holes opening zone 11, the three holes are arranged to define a triangular shape within the three dimensional space formed on wall of the intraluminal main trachea tube 1; the triangular shape as defined can be an equilateral triangle which means all the three included angles thereof are 60 degrees.

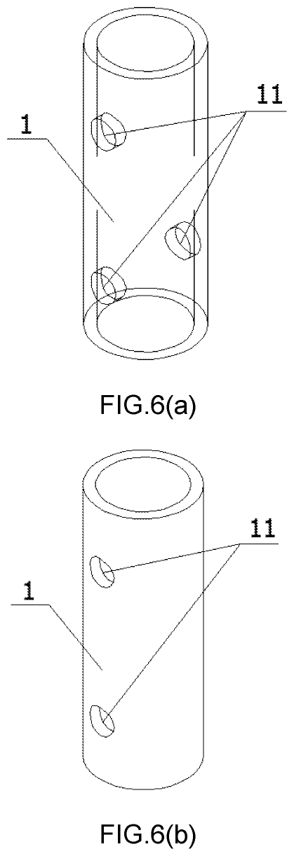

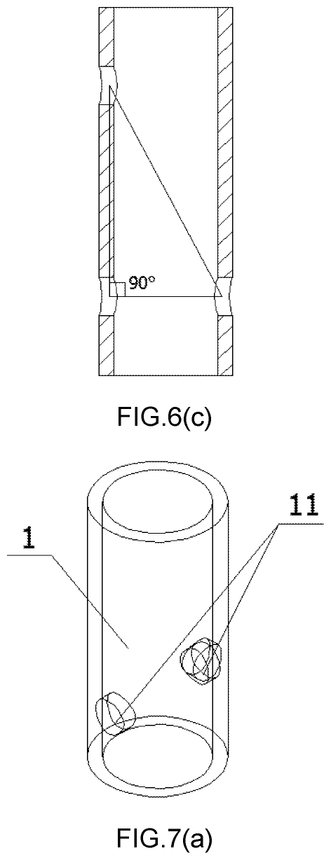

FIGS. 6(a), 6(b) and 6(c) are perspective and vertical sectional views showing a fifth embodiment of holes distribution of the second holes opening zone according to an embodiment of the present invention.

As shown in FIGS. 6(a), 6(b) and 6(c), when there are three holes in the second holes opening zone 11, the three holes are arranged to define a triangular shape within the three dimensional space formed on wall of the intraluminal main trachea tube 1; the triangular shape as defined can be a right angled triangle which means one of the three included angles thereof is 90 degrees.

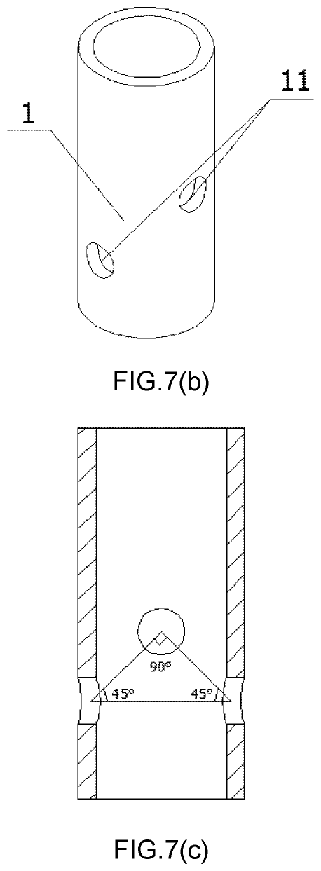

FIGS. 7(a), 7(b) and 7(c) are perspective and vertical sectional views showing a sixth embodiment of hole distribution of the second holes opening zone according to an embodiment of the present invention.

As shown in FIGS. 7(a), 7(b) and 7(c), when there are three holes in the second holes opening zone 11, the three holes are arranged to define a triangular shape within the three dimensional space formed on wall of the intraluminal main trachea tube 1; the triangular shape as defined can be a right angled isosceles triangle which means one of the three included angles thereof is 90 degrees while the two other included angles are each 45 degrees.

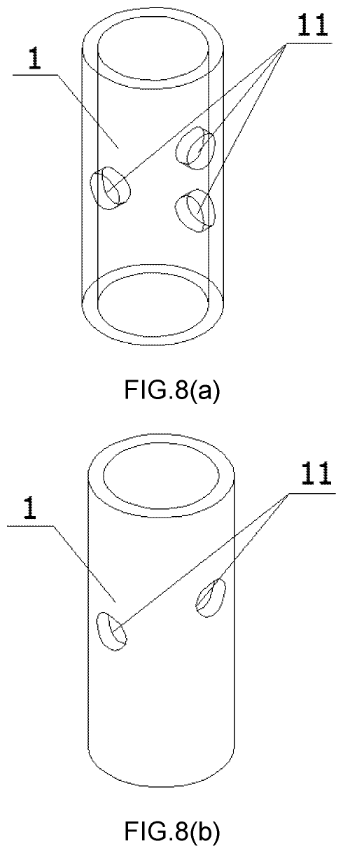

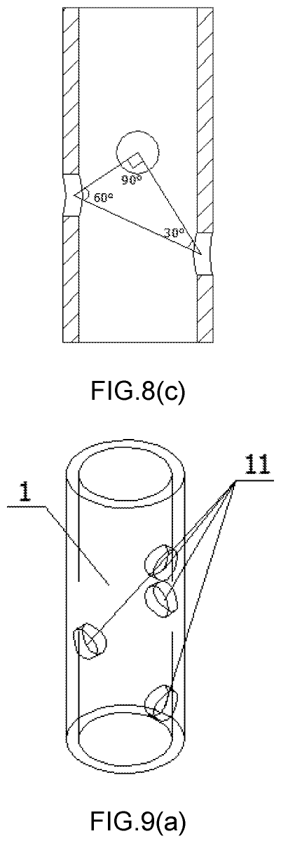

FIGS. 8(a), 8(b) and 8(c) are perspective and vertical sectional views showing a seventh embodiment of holes distribution of the second holes opening zone according to an embodiment of the present invention.

As shown in FIGS. 8(a), 8(b) and 8(c), when there are three holes in the second holes opening zone 11, the three holes are arranged to define a triangular shape within the three dimensional space formed on wall of the intraluminal main trachea tube 1; the triangular shape as defined can be a right angled triangle having an included angle of 30 degrees, which means one of the three included angles thereof is 30 degrees, the second included angle is 60 degrees, and the last included angle is 90 degrees.

Obviously, apart from the above specified types of different triangular shapes, other triangular shapes each having included angles of different degrees may also be possible and those other triangular shapes should also fall within the scope of protection of the present invention.

As shown in FIG. 3(a)-FIG. 3(c), when there are four holes in the second holes opening zone 11, the four holes are arranged to define a parallelogram shape within the three dimensional space formed by an inner side wall of the intraluminal main trachea tube 1.

Alternatively, as shown in FIG. 4(a)-FIG. 4(c), when there are four holes in the second holes opening zone 11, the four holes divided in two pairs with each pair of holes opposing each other define a cross shape within the three dimensional space formed on wall of the intraluminal main trachea tube 1.

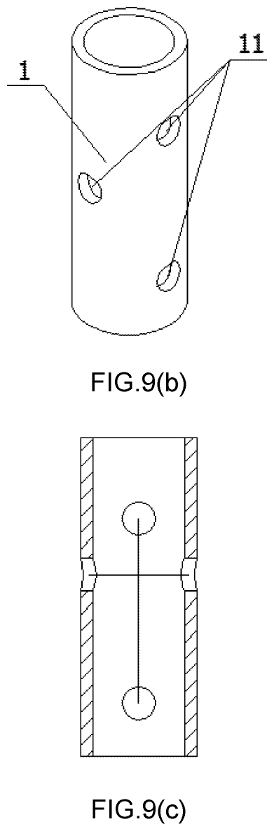

FIGS. 9(a), 9(b) and 9(c) are perspective and vertical sectional views showing an eighth embodiment of holes distribution of the second holes opening zone according to an embodiment of the present invention.

Further as shown in FIGS. 9(a), 9(b) and 9(c), when there are four holes in the second holes opening zone 11, the four holes divided in two pairs with each pair of holes opposing each other define a cross shape within the three dimensional space formed by an inner side wall of the intraluminal main trachea tube 1, and the cross shape has equal width and height. Of course, the cross shape may not have equal width and height.

It should also be noted that the quantity and the specific arrangement of holes of the second holes opening zone should not be limited to the above specified embodiments. Other reasonable arrangements may be possible, provided that the strength of the wall of the intraluminal main trachea tube 1 is ensured to prevent the present invention from damage due to bending. For example, there may be four holes in the second holes opening zone, and the four holes are arranged to define a tetrahedral shape within the three dimensional space formed by an inner side wall of the intraluminal main trachea tube 1; or there may be five holes in the second holes opening zone, and the five holes are arranged to define a pentagonal shape within the three dimensional space formed by an inner side wall of the intraluminal main trachea tube 1; or there may be six holes in the second holes opening zone, and the six holes are arranged to define a pentahedral shape within the three dimensional space formed by an inner side wall of the intraluminal main trachea tube 1, so and so forth, without limitation to the above described examples.

During actual use of the present invention, each of the holes through the wall of the intraluminal main trachea tube in the second holes opening zone 11 is inclined. Specifically, each hole is inclined with respect to a perpendicular position to a side wall of the intraluminal main trachea tube 1; since the holes are inclined, each hole is inclined with respect to a perpendicular position to a side wall of the intraluminal main trachea tube 1, and as such, the gas flow resembles more to physiological mixed flow of gas based on the angle of inclination and the amount of gas of each divided gas path. Accordingly, the ventilated gas can be more converged and focused and thereby enhancing the effect of the use of the present invention.

In particular, the wall of each hole is inclined with respect to a perpendicular position to a side wall of the intraluminal main trachea tube 1, and an angle of inclination is 0-90 degrees in wall of the trachea tube.

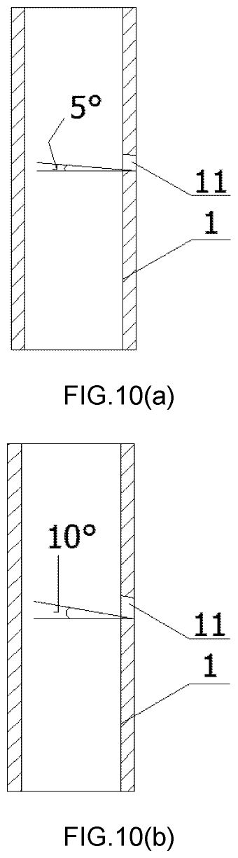

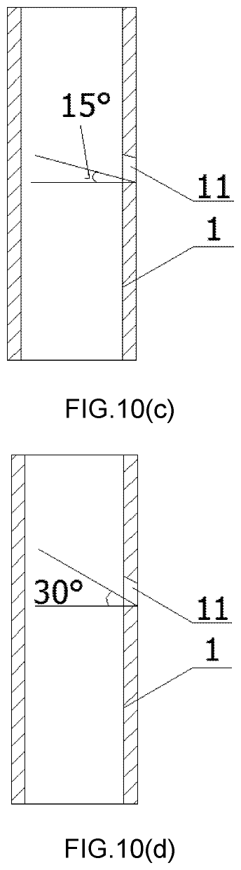

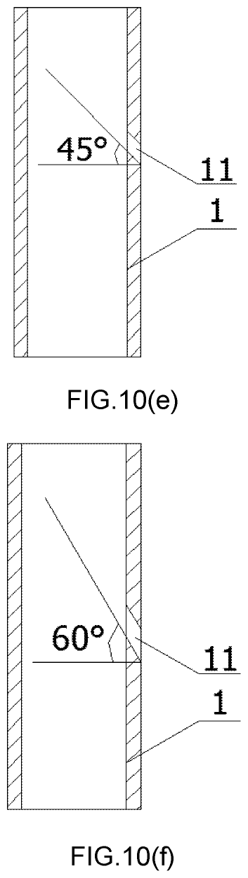

FIG. 10(a)-FIG. 10(f) are sectional views showing different possible angles of holes through the wall of the tube of the second holes opening zone according to the present invention.

To ensure a more converged and focused flow of ventilated gas flowing of wall along the second hole opening zone 11, and to facilitate processing and manufacturing, the possible angles of inclination can be for example the angle of holes through the wall of the tube 5, 10, 15, 30, 45 or 60 degrees in wall of the trachea tube, as shown in FIG. 10(a)-FIG. 10(f). Of course, the angle of inclination should not be limited to the given examples. Without limitation, the angle of inclination can be any angle of hole through the wall of the tube between 0-90 degrees.

Also, as shown in FIG. 10(a)-FIG. 10(f), in order to achieve better ventilation, an end of each hole at an inner side wall of the second holes opening zone of the intraluminal main trachea tube 1 is positioned higher than another end thereof at an outer side wall of the second holes opening zone of the intraluminal main trachea tube 1.

In actual use of the present invention, a suction hole 12 can be provided at a lower part of the intraluminal main trachea tube 1 proximal to the distal intraluminal bronchus tube 2, as shown in FIG. 1, to facilitate phlegm suction. The suction hole 12 faces away from the first opening 21. Preferably, a hole diameter of the suction hole 12 is the same as a diameter of the first opening 21.

It should be noted that, the suction hole 12 is not preferred to have an overly small size which may result in failed connection of the suction hole 12 with a specific suction catheter and thereby resulting in poor suction effect. However, the suction hole 12 should not be too large which weakens the mechanical strength of the present invention, causing the present invention to be broken and left inside the lung of the patient.

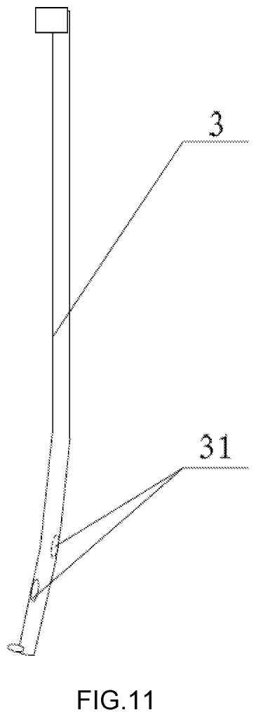

FIG. 11 shows the structure of a suction catheter specifically to be used with the present invention.

With reference to FIG. 1 and FIG. 11, the suction catheter 3 may be inserted into the suction hole 12; two small holes 31 are provided on the suction catheter 3; the two small holes 31 can be opposed to each other for balancing pressure.

Specifically, to facilitate insertion of the suction catheter 3 into the present invention, one end of the suction catheter 3 can be bent, and an angle of bending is preferably 90-150 degrees.

Further, in actual production, the angle of bending of said one end of the suction catheter 3 can be 90, 120 or 150 degrees. Of course, the angle of bending should not be limited to the given examples. Without limitation, other reasonable angles are possible.

Further, to ensure good suction effect and to facilitate insertion and removal of the suction catheter 3, the suction catheter 3 has a diameter preferably of one third of the diameter of the distal intraluminal bronchus tube 2.

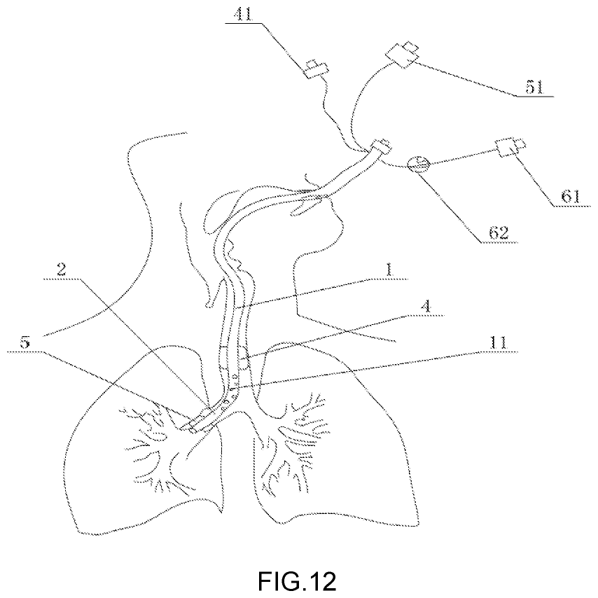

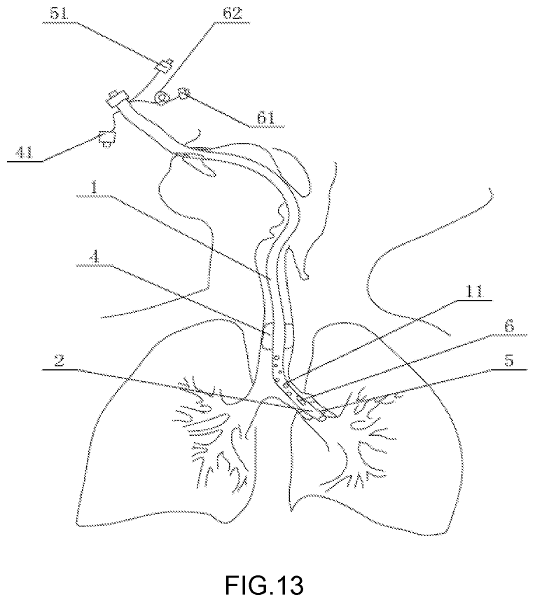

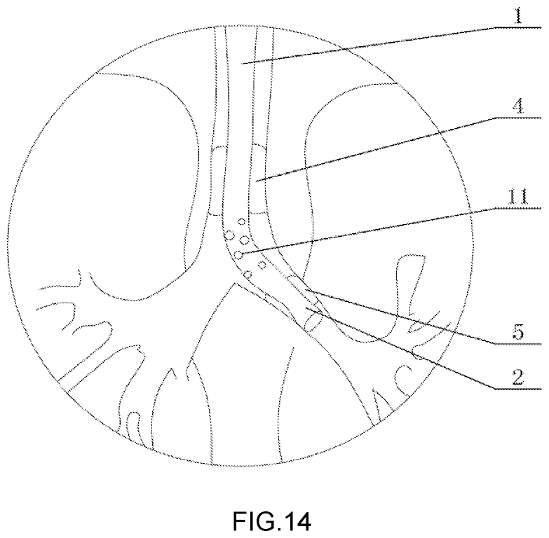

FIG. 12 is a diagram showing the use of the present invention. FIG. 13 is another diagram showing the use of the present invention. FIG. 14 shows the bridge structure spans and balanced lung ventilation of the present invention during use.

As shown in FIG. 1 and FIG. 12-FIG. 14, during the actual use of the present invention, an outer wall of the intraluminal main trachea tube 1 at a position above the second holes opening zone 11 is provided with a tracheal cuff 4; the tracheal cuff 4 is in communication with a first inflating valve 41; an outer wall of the distal intraluminal bronchus tube 2 is provided with a bronchial cuff 5; the bronchial cuff 5 is in communication with a second inflating valve 51; a distal intraluminal balloon blocker 6 is provided inside the distal intraluminal bronchus tube 2; the distal intraluminal balloon blocker 6 is in communication with a third inflating valve 61 via a ventilation pressure controller 62. The ventilation pressure controller 62 indicates pressure level inside the distal intraluminal balloon blocker 6 and adjusts a size of the distal intraluminal balloon blocker 6 for blocking, such that an amount of ventilated gas flow at the distal intraluminal bronchus tube can be regulated and the balanced ventilation between the left and right lungs can be regulated.

The present invention has the following beneficial effects:

1. The present invention has all the functions of a conventional double-lumen endotracheal tube, while overcoming the disadvantages of the conventional double-lumen endotracheal tube including the difficulty of inserting into the trachea and the problem of causing tracheal injury.

2. During operation, the tube may displace. Provision of the second holes opening zone on the tube according to the present invention prevents poor ventilation caused by blocking of a second hole of the tube due to rotation of the tube during operation.

3. The second holes opening zone allows the coverage area of holes to be configured practically and scientifically, thus preventing bending of the tube and increasing the stability of the tube, thereby solving the existing problem of easy bending of the tube having a single hole because of the excessively large diameter of the hole.

4. After the tracheal cuff and the bronchial cuff are inflated, two ends of each cell bulge such that the opening is supported at central parts of the trachea and the bronchus away from the inner walls of the trachea and the bronchus by the bulged ends of the air cells to form the so called bridge structure spans. During operation, the bridge structure spans can prevent rotation and displacement of the tube due to positional changes of the patient's body, and can prevent the opening of the tube from touching the wall of the trachea and bronchus. Besides, due to the multiple holes in the second holes opening zone, air can be divided into different paths and thus reducing impact force of air and avoiding air advection. The multiple holes together with the air cells can effectively prevent the tube from displacement.

5. The suction catheter is facilitated to be inserted for easier phlegm suction. The second holes opening zone may be selected for a double opening tube to perform suction.

6. The distal intraluminal balloon blocker is a balloon shaped air cell embedded into the wall of the tube. The distal intraluminal balloon blocker is connected with the third inflating valve and also the ventilation pressure controller which can indicate the pressure inside the distal intraluminal balloon blocker and can regulate the size of the distal intraluminal balloon blocker. Accordingly, it achieves the purposes of regulating the amount of ventilated air at the bronchus and regulating the balanced ventilation between the left and right lungs, thereby meeting the requirements for surgeries that require extended operation time, ensuring a controlled lung collapse area at the operation site, ensuring the most preferred spatial requirements for surgery, and preventing acute lung injury, recurrent pulmonary edema, and general lung infections after surgery. The advantage herein achieved is so called controlled balance ventilation.

It should be noted that, in the present invention as shown in FIG. 1-FIG. 14, the intraluminal main trachea tube 1, the distal intraluminal bronchus tube 2 and the suction catheter 3 all have circular cross sections. The first opening 21, the holes of the second holes opening zone 11 and the small holes 31 can be circular in shape. Of course, without limitation, the small holes can have other shapes.

The first opening 21 has an area S_{end}; a total area of the holes in the second holes opening zone 11 is S; wherein S≥1.5S_{end}.

For example, there are two holes in the second holes opening zone 11, and the holes have circular or elliptical shapes, and a total area of the holes is determined according to the principles of aerodynamics to achieve controlled balanced ventilation in the lung, wherein S1+S2≥1.5S_{end}. The left lung and the right lung have balanced tidal volume (TV) of ventilated air meeting physiological standards. Given a patient of 60 kg, the TV of the left lung is TV_{L}, and the TV of the right lung is TV_{R}, then TV_{L}=TV_{R}=200-370 ml. By means of the holes that achieve balanced ventilation in the lung to attain S1+S2≥1.5S_{end}, balanced ventilation between the left lung and the right lung is achieved. The same principles apply to different body weights.

In another example, there are three holes in the second holes opening zone 11, and a total area of the holes is determined according to the principles of aerodynamics to achieve controlled balanced ventilation in the lung, wherein S1+S2+S3≥1.5S_{end}. The left lung and the right lung have balanced tidal volume (TV) of ventilated air meeting physiological standards. Given a patient of 60 kg, the TV of the left lung is TV_{L}, and the TV of the right lung is TV_{R}, then TV_{L}=TV_{R}=200-370 ml. By means of the holes that achieve balanced ventilation in the lung to attain S1+S2+S3≥1.5S_{end}, balanced ventilation between the left lung and the right lung is achieved. The same principles apply to different body weights.

In another example, there are four holes in the second holes opening zone 11, and a total area of the holes is determined according to the principles of aerodynamics to achieve controlled balanced ventilation in the lung, wherein S1+S2+S3+S4≥1.5S_{end}. The left lung and the right lung have balanced tidal volume (TV) of ventilated air meeting physiological standards. Given a patient of 60 kg, the TV of the left lung is TV_{L}, and the TV of the right lung is TV_{R}, then TV_{L}=TV_{R}=200-370 ml. By means of the holes that achieve balanced ventilation in the lung to attain S1+S2+S3+S4≥2.5S_{end}, balanced ventilation between the left lung and the right lung is achieved. The same principles apply to different body weights.

Further examples having more than four holes in the second holes opening zone 11 and other configurations of holes apply the same principles and will not be further discussed herein.

The present invention provides and increases the efficiency of balanced ventilation between the left lung and the right lung achieved by a novel lung isolation catheter during thoracic surgery. The geometric arrangements of the holes on the wall of the present invention solve the problems of damaging of the tube due to bending that shortens its service life, and also increase the mechanical supportive strength of the tube at the second holes opening zone. Combined use of the above two technical features of the present invention enables the ventilation provided by the present invention to resemble more to the respiration physiology of a patient, strengthen the parts provided with holes, and increase the adaptability of the tube.

An object of the present invention is to substitute the double-lumen endotracheal tube which is being currently used. The theoretical basis of the present invention involves controlled balanced ventilation (CBV) and bridge structure span (BSS). The present invention aims at providing a tube of practical utility, such a tube can ensure effective balanced ventilation during clinically anaesthetized lung isolation, control the lung collapse or deflation at the target lung, and can simplify alignment procedure by a bridge structure span that supports the present invention at the centers of the trachea and the bronchus away from the inner walls of the trachea and the bronchus by bulged portions; further, the tube diameter can be reduced and the cross sectional area for ventilation can be increased when compared with a conventional tube. The present invention substitutes and even expands the functions of the conventional double-lumen endotracheal tube, satisfying the requirements for lung isolation in children and improve ventilation of special patients like those who suffer from chronic obstructive pulmonary disease (COPD) during anaesthetized surgery.

Only the preferred embodiments of the present invention are described above. However, these described embodiments should not limit the present invention. Any changes, modifications, or alternative configurations achieving the same technical effects according to the concept and spirit of the present invention should also fall within the scope of protection of the present invention.

1. A medical mechanical controlled balance ventilation single-lumen lung isolation catheter bridging spans trachea to bronchus and by-pass flow gas holes of wall, comprising a intraluminal main trachea tube and a distal intraluminal bronchus tube in communication with one end of the intraluminal main trachea tube; a first opening is provided at a rear end of the distal intraluminal bronchus tube; a second holes opening zone having at least two holes is provided at a side wall of a lower end of the intraluminal main trachea tube; the holes of the second holes opening zone are distributed along a same or different horizontal sectional surface or vertical sectional surface of a three dimensional space formed inside the intraluminal main trachea tube.

2. The medical mechanical controlled balance ventilation single-lumen lung isolation catheter as in claim 1, wherein when the second holes opening zone has three holes, the three holes are arranged to define a triangular shape within the three dimensional space formed on wall of the intraluminal main trachea tube.

3. The medical mechanical controlled balance ventilation single-lumen lung isolation catheter as in claim 2, wherein when the second holes opening zone has three holes, and the three holes are arranged to define the triangular shape within the three dimensional space formed on wall of the intraluminal main trachea tube, the triangular shape as defined is an isosceles triangle.

4. The medical mechanical controlled balance ventilation single-lumen lung isolation catheter as in claim 2, wherein when the second holes opening zone has three holes, and the three holes are arranged to define the triangular shape within the three dimensional space formed on wall of the intraluminal main trachea tube, the triangular shape as defined is an equilateral triangle.

5. The medical mechanical controlled balance ventilation single-lumen lung isolation catheter as in claim 2, wherein when the second holes opening zone has three holes, and the three holes are arranged to define the triangular shape within the three dimensional space formed on wall of the intraluminal main trachea tube, the triangular shape as defined is a right angled triangle.

6. The medical mechanical controlled balance ventilation single-lumen lung isolation catheter as in claim 2, wherein when the second holes opening zone has three holes, and the three holes are arranged to define the triangular shape within the three dimensional space formed on wall of the intraluminal main trachea tube, the triangular shape as defined is a right angled isosceles triangle.

7. The medical mechanical controlled balance ventilation single-lumen lung isolation catheter as in claim 2, wherein when the second holes opening zone has three holes, and the three holes are arranged to define the triangular shape within the three dimensional space formed on wall of the intraluminal main trachea tube, the triangular shape as defined is a right angled triangle having one included angle of 30 degrees.

8. The medical mechanical controlled balance ventilation single-lumen lung isolation catheter as in claim 2, wherein when the second holes opening zone has three holes, and the three holes are arranged to define the triangular shape within the three dimensional space formed on wall of the intraluminal main trachea tube, the triangular shape as defined has 180 degrees being a sum of all included angles.

9. The medical mechanical controlled balance ventilation single-lumen lung isolation catheter as in claim 1, wherein when there are four holes in the second holes opening zone, the four holes are arranged to define a parallelogram shape or any quadrilateral within the three dimensional space formed on wall of the intraluminal main trachea tube.

10. The medical mechanical controlled balance ventilation single-lumen lung isolation catheter as in claim 1, wherein when there are four holes in the second holes opening zone, the four holes divided into two pairs with each pair of holes opposing each other define a cross shape or any diagonal shape within the three dimensional space formed on wall of the intraluminal main trachea tube.

11. The medical mechanical controlled balance ventilation single-lumen lung isolation catheter as in claim 10, wherein when there are four holes in the second holes opening zone, the four holes divided into two pairs with each pair of holes opposing each other define the cross shape within the three dimensional space formed on wall of the intraluminal main trachea tube, the cross shape as defined has equal width and height or the cross shape as defined has unequal width and height.

12. The medical mechanical controlled balance ventilation single-lumen lung isolation catheter as in claim 1, wherein each of the holes of the second holes opening zone is an inclined hole through the wall of the intraluminal main trachea tube; each hole through the wall of the intraluminal main trachea tube is inclined with respect to a perpendicular position to a side wall of the intraluminal main trachea tube.

13. The medical mechanical controlled balance ventilation single-lumen lung isolation catheter as in claim 12, wherein each of the holes of the second holes opening zone is the inclined hole through the wall of the intraluminal main trachea tube; the wall of each hole through the wall of the intraluminal main trachea tube is inclined with respect to the perpendicular position to the side wall of the intraluminal main trachea tube, wherein an angle of inclination of each hole is 0-90 degrees.

14. The medical mechanical controlled balance ventilation single-lumen lung isolation catheter as in claim 13, wherein the angle of inclination of each hole through the wall of the intraluminal main trachea tube is 5 degrees.

15. The medical mechanical controlled balance ventilation single-lumen lung isolation catheter as in claim 13, wherein the angle of inclination of each hole through the wall of the intraluminal main trachea tube is 10 degrees.

16. The medical mechanical controlled balance ventilation single-lumen lung isolation catheter as in claim 13, wherein the angle of inclination of each hole through the wall of the intraluminal main trachea tube is 15 degrees.

17. The medical mechanical controlled balance ventilation single-lumen lung isolation catheter as in claim 13, wherein the angle of inclination of each hole through the wall of the intraluminal main trachea tube is 30 degrees.

18. The medical mechanical controlled balance ventilation single-lumen lung isolation catheter as in claim 13, wherein the angle of inclination of each hole through the wall of the intraluminal main trachea tube is 45 degrees.

19. The medical mechanical controlled balance ventilation single-lumen lung isolation catheter as in claim 13, wherein the angle of inclination of each hole through the wall of the intraluminal main trachea tube is 60 degrees.

20. The medical mechanical controlled balance ventilation single-lumen lung isolation catheter as in claim 1, wherein a suction hole is provided at a lower part of the intraluminal main trachea tube proximal to the distal intraluminal bronchus tube; the suction hole faces away from the first opening; a hole diameter of the suction hole is the same as a diameter of the first opening.

21. The medical mechanical controlled balance ventilation single-lumen lung isolation catheter as in claim 20, wherein a suction catheter is inserted into the suction hole; two small holes are provided on the suction catheter; the two small holes are opposed to each other.

22. The medical mechanical controlled balance ventilation single-lumen lung isolation catheter as in claim 21, wherein one end of the suction catheter is bent, and an angle of bending is 90-150 degrees.

23. The medical mechanical controlled balance ventilation single-lumen lung isolation catheter as in claim 21, wherein the suction catheter has a diameter of one third of a diameter of the distal intraluminal bronchus tube.

24. The medical mechanical controlled balance ventilation single-lumen lung isolation catheter as in claim 1, wherein an outer wall of the intraluminal main trachea tube at a position above the second holes opening zone is provided with a tracheal cuff; the tracheal cuff is in communication with a first inflating valve;
an outer wall of the distal intraluminal bronchus tube is provided with a bronchial cuff; the bronchial cuff is in communication with a second inflating valve;
a distal intraluminal balloon blocker is provided inside the distal intraluminal bronchus tube; the distal intraluminal balloon blocker is in communication with a third inflating valve via a ventilation pressure controller;
the ventilation pressure controller indicates pressure level inside the distal intraluminal balloon blocker and adjusts a size of the distal intraluminal balloon blocker for blocking, such that an amount of ventilated air flow at the distal intraluminal bronchus tube is regulated and a balanced ventilation between left and right lungs is regulated.

25. The medical mechanical controlled balance ventilation single-lumen lung isolation catheter as in claim 24, wherein the first opening has an area S_{end}; a total area of the holes in the second holes opening zone is S; wherein S≥1.5S_{end}.

26. The medical mechanical controlled balance ventilation single-lumen lung isolation catheter as in claim 6, wherein bronchial Sealing airbag inside the bronchus tube with adjustable size.
